(19) United States
(12) Patent Application Publication
(10) Pub. No.: US 2024/0408262 A1
(43) Pub. Date: Dec. 12, 2024

(54) PURE ESSENTIAL OIL ATOMIZATION DEVICE

(71) Applicants: AUDIOWELL ELECTRONICS (GUANGDONG) CO., LTD., Guangzhou (CN); GUANGZHOU AUDIOWELL SENSING APPLICATION CO., LTD., Guangzhou (CN)

(72) Inventors: Hengrui Zheng, Guangzhou (CN); Weipei Liang, Guangzhou (CN); Zhuocong Xu, Guangzhou (CN); Bo Peng, Guangzhou (CN)

(21) Appl. No.: 18/385,906

(22) Filed: Oct. 31, 2023

(30) Foreign Application Priority Data
Jun. 9, 2023 (CN) 202310682163.1

(51) Int. Cl. A61L 9/14

(52) U.S. Cl. CPC A61L 9/14; A61L 2209/132; A61L 2209/133

(57) ABSTRACT

A pure essential oil atomization device includes a bottle body, a housing, a guide cotton, and an ultrasonic atomization sheet, where the bottle body is provided therein with a pure essential oil chamber, and is provided with an opening communicated with the pure essential oil chamber; the housing is detachably connected to the bottle body, and covers the opening; the housing is provided therein with an atomizing chamber, and is provided with an atomization outlet communicated with the atomizing chamber; the ultrasonic atomization sheet is provided in the atomizing chamber, and includes an atomizing plane; and the guide cotton includes one end attached to the atomizing plane and surrounding a center of the atomizing plane, and the other end extending through the housing to the pure essential oil chamber. The present disclosure effectively solves the problems of leakage and noise in the prior art.

### CROSS REFERENCE TO RELATED APPLICATIONS

The present application claims the benefit of Chinese Patent Application No. 202310682163.1 filed on Jun. 9, 2023, the contents of which are incorporated herein by reference in their entirety.

### TECHNICAL FIELD

The present disclosure relates to the technical field of atomization devices, and in particular to a pure essential oil atomization device.

### BACKGROUND

At present, pure essential oil atomization devices usually use atomization methods such as microporous atomization, compression pump atomization, and water mixed aromatherapy atomization. In the microporous atomization method, a microporous atomizer comes into direct contact with the pure essential oil, and vibrates and compresses the pure essential oil to achieve the purpose of atomization. In the compression pump atomization method, the pure essential oil is dispersed by high-speed air obtained by compressing the air to achieve the effect of atomization and aromatherapy. In the water mixed aromatherapy atomization method, the pure essential oil is dropped into or dissolved in water and form a water-oil mixture, which is atomised by ultrasound, thereby diffusing essential oil particles in the water into the air.

The microporous atomization method has problems such as leakage and short service life, and cannot atomize high-viscosity pure essential oils. Although the compression pump atomization method can atomize high-viscosity pure essential oils, the compression pump used in it has problems such as complex structure, large volume, and high working noise. The water mixed aromatherapy atomization method has the problem of pure essential oil contamination.

Therefore, the current pure essential oil atomization devices need improvement.

### SUMMARY

An objective of the present disclosure is to design a pure essential oil atomization device, so as to solve the aforementioned problems.

In order to achieve the above objective, the present disclosure provides a pure essential oil atomization device, including a bottle body, a housing, a guide cotton, and an ultrasonic atomization sheet, where
the bottle body is provided therein with a pure essential oil chamber, and is provided with an opening communicated with the pure essential oil chamber; the housing is detachably connected to the bottle body, and covers the opening; the housing is provided therein with an atomizing chamber, and is provided with an atomization outlet communicated with the atomizing chamber; the ultrasonic atomization sheet is provided in the atomizing chamber, and includes an atomizing plane; and the guide cotton includes one end attached to the atomizing plane and surrounding a center of the atomizing plane, and the other end extending through the housing to the pure essential oil chamber.

Preferably, the atomizing plane is circular with a radius of R mm; and a maximum distance of L mm and a minimum distance of G mm between projection of the guide cotton on the atomizing plane and the center of the atomizing plane, 0.3≤G/R≤L/R≤0.9.

Preferably, projection of the guide cotton on the atomizing plane surrounds the center of the atomizing plane, and a central angle of an arc of the projection is N°, 180≤N≤360.

Preferably, an atomization temperature of the ultrasonic atomization sheet is T° C., 80≤T≤160.

Preferably, the guide cotton includes a first guide cotton and a second guide cotton connected to each other; the first guide cotton is attached to the atomizing plane, and the second guide cotton extends through the housing to the pure essential oil chamber; and the first guide cotton is made of a high temperature resistance material.

Preferably, the second guide cotton is hollow.

Preferably, the opening of the bottle body is oriented upwards; the housing covers the opening from top to bottom and is screwed to the bottle body; and a bottom part of the housing is provided with an air hole that communicates the atomizing chamber with the pure essential oil chamber.

Preferably, the housing is further provided therein with an air chamber, and is further provided with an air inlet communicated with the air chamber; the atomizing chamber is communicated with the atomization outlet through the air chamber; and the atomizing plane is oriented towards the air chamber.

Preferably, a cross-section of a wall of the air chamber is circular.

Preferably, the atomization outlet is provided at a top part of the housing; the air inlet is provided at a side wall of the housing; and the air inlet and the atomization outlet are respectively arranged on left and right sides of the housing.

Compared with the prior art, the pure essential oil atomization device provided by an embodiment of the present disclosure has the following beneficial effects:

In the embodiment of the present disclosure, the pure essential oil atomization device uses the ultrasonic atomization sheet to directly atomize the pure essential oil, effectively solving the problems of leakage and noise in the prior art. In addition, the pure essential oil atomization device uses the guide cotton to guide the pure essential oil, avoiding direct contact between the pure essential oil and the ultrasonic atomization sheet. The pure essential oil is laid flat on the atomizing plane of the ultrasonic atomization sheet, thereby enhancing the atomization effect and improving the atomization efficiency.

### BRIEF DESCRIPTION OF THE DRAWINGS

FIG. 1 is a structural diagram of a pure essential oil atomization device according to an embodiment of the present disclosure;

FIG. 2 is another structural diagram of the pure essential oil atomization device according to the embodiment of the present disclosure;

FIG. 3 is a plane view of an atomizing plane and a guide cotton of a pure essential oil atomization device according to an embodiment of the present disclosure;

FIG. 4 is a plane view of an atomizing plane and a guide cotton of a pure essential oil atomization device according to an embodiment of the present disclosure;

FIG. 5 is a plane view of an atomizing plane and a guide cotton of a pure essential oil atomization device according to an embodiment of the present disclosure;

FIG. 6 is a plane view of an atomizing plane and a guide cotton of a pure essential oil atomization device according to an embodiment of the present disclosure;

FIG. 7 is a structural diagram of a pure essential oil atomization device according to an embodiment of the present disclosure; and

FIG. 8 is a structural diagram of a pure essential oil atomization device according to an embodiment of the present disclosure.

Reference Signs: 1: bottle body; 11: pure essential oil chamber; 12: opening; 2: housing; 21: atomizing chamber; 22: atomization outlet; 23: air hole; 24: air chamber; 25: air inlet; 3: guide cotton; 31: first guide cotton; 32: second guide cotton; 4: ultrasonic atomization sheet; and 41: atomizing plane.

### DETAILED DESCRIPTION

The specific implementations of the present disclosure are described in more detail below with reference to the drawings and embodiments. The following embodiments are illustrative of the present disclosure and should not be construed as limiting of the scope of the present disclosure.

It should be understood that in the description of the present disclosure, orientation or position relationships indicated by terms such as “upper”, “lower”, “front”, “rear”, “left”, “right”, “vertical”, “horizontal”, “top”. “bottom”, “inner”, and “outer” are orientation or position relationships as shown in the drawings. These terms are merely intended to facilitate and simplify the description of the present disclosure, rather than to indicate or imply that the mentioned device or component must have a specific orientation or must be constructed and operated in a specific orientation. Therefore, these terms should not be understood as a limitation to the present disclosure.

In the description of the present disclosure, unless otherwise clearly defined, the terms such as “interconnection”, “connection”, and “fixation” are intended to be understood in a broad sense. For example, the “connection” may be a fixed connection, a removable connection or an integral connection; may be a mechanical connection or a welded connection; may be a direct connection or an indirect connection via an intermediate medium; and may be a communication or interaction between two elements. Those of ordinary skill in the art may understand specific meanings of the above terms in the present disclosure based on a specific situation.

In addition, the terms such as “first” and “second” are used in the present disclosure to describe various kinds of information, but the information should not be limited to these terms, and these terms are only used to distinguish the same type of information from each other. For example, without departing from the scope of the present disclosure, “first” information may be referred to as “second” information, and similarly, “second” information may also be referred to as “first” information.

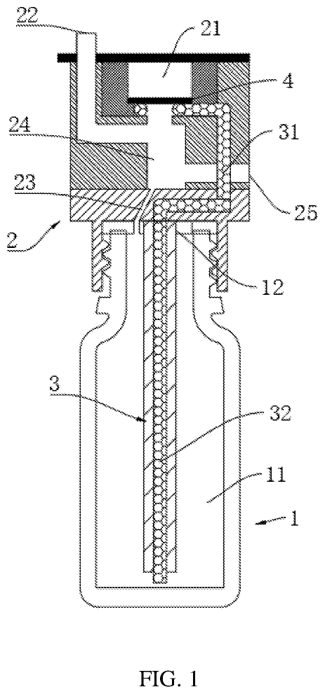

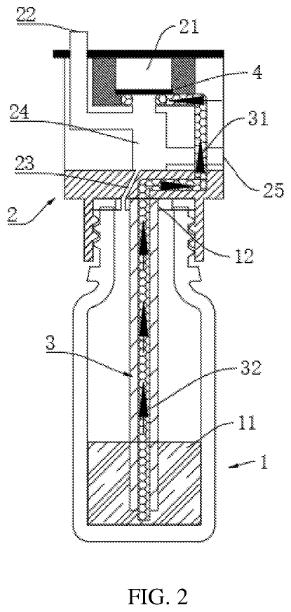

As shown in FIGS. 1 and 2, a preferred embodiment of the present disclosure provides a pure essential oil atomization device, including a bottle body 1, a housing 2, a guide cotton 3, and an ultrasonic atomization sheet 4.

The bottle body 1 is provided therein with a pure essential oil chamber 11, and the bottle body 1 is provided with an opening 12 communicated with the pure essential oil chamber 11. The housing 2 is detachably connected to the bottle body 1, and the housing 2 covers the opening 12. The housing 2 is provided therein with an atomizing chamber 21, and the housing 2 is provided with an atomization outlet 22 communicated with the atomizing chamber 21. The ultrasonic atomization sheet 4 is provided in the atomizing chamber 21, and the ultrasonic atomization sheet 4 is provided with an atomizing plane 41. One end of the guide cotton 3 is attached to the atomizing plane 41, and the one end of the guide cotton 3 surrounds a center of the atomizing plane 41, which refers to a geometric center of the atomizing plane. The other end of the guide cotton 3 extends through the housing 2 to the pure essential oil chamber 11.

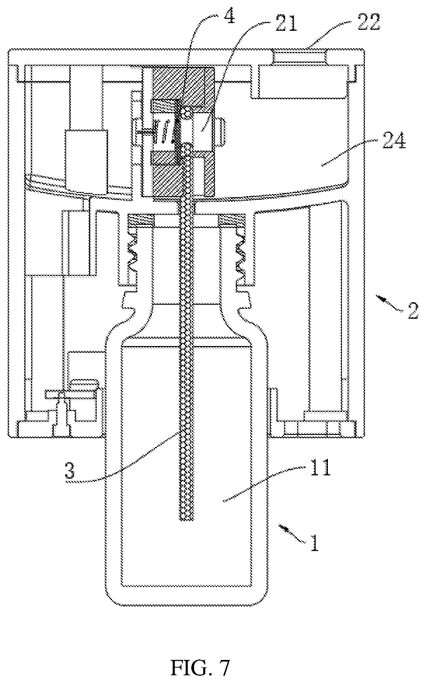

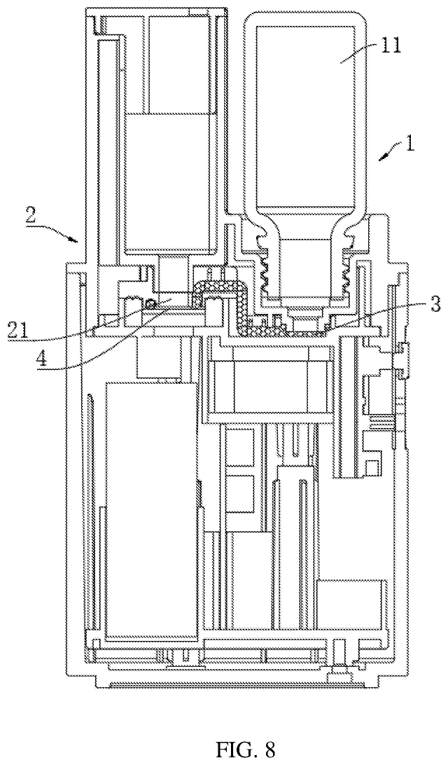

In this solution, the ultrasonic atomization sheet 4 directly atomizes the pure essential oil. The pure essential oil atomization device needs to avoid direct contact between the pure essential oil and the ultrasonic atomization sheet 4, so as to avoid the problem of poor atomization efficiency and poor atomization effect caused by uneven pure essential oil supply. For this purpose, the guide cotton 3 serves as a pure essential oil passage to guide the pure essential oil in the pure essential oil chamber 11 to come into contact with the atomizing plane 41. The design enhances the atomization effect, improves the atomization efficiency, and effectively solves problems such as leakage and noise during use. As shown in FIGS. 1, 7, and 8, the ultrasonic atomization sheet 4 can be provided in any manner, such as inverted (FIG. 1), vertical (FIG. 7), or horizontal (FIG. 8). In the inverted manner, a contact surface of the ultrasonic atomization sheet with the pure essential oil is oriented downwards. In the horizontal manner, the contact surface of the ultrasonic atomization sheet with the pure essential oil is oriented upwards. The placement manners vary to suit different device usage scenarios, thereby enriching device diversity.

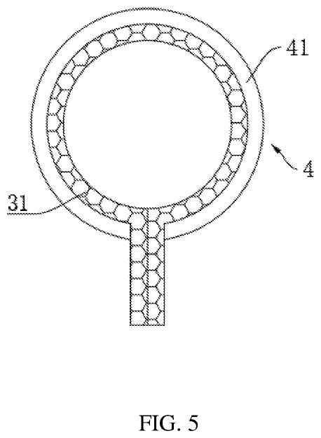

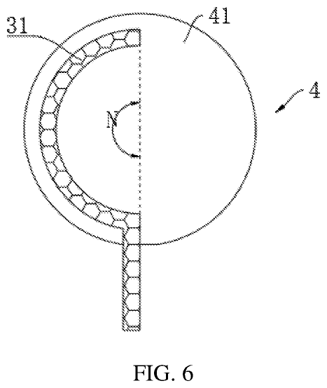

As shown in FIGS. 5 and 6, in some improvement solutions of the present disclosure, projection of the guide cotton 3 on the atomizing plane 41 surrounds the center of the atomizing plane 41, and a central angle of an arc of the projection is N°, 180≤N≤360. The one end of the guide cotton 3 is tightly attached to the atomizing plane 41 of the ultrasonic atomization sheet 4 in a semi-circular or full-circular manner, and the other end of the guide cotton 3 is in contact with the pure essential oil in the pure essential oil chamber 11. Through the pure essential oil passage, the pure essential oil in the bottle body 1 is introduced into the ultrasonic atomization sheet 4, avoiding direct contact between the pure essential oil and the ultrasonic atomization sheet 4.

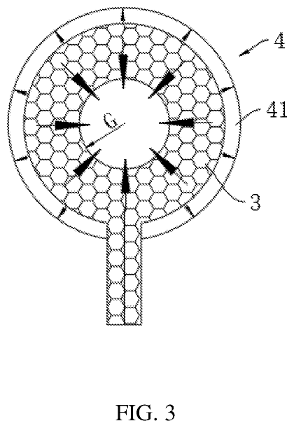

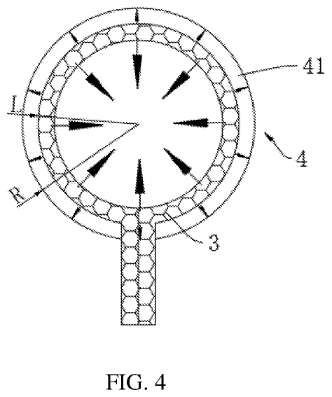

As shown in FIGS. 3 and 4, in some improvement solutions of the present disclosure, the atomizing plane 41 is circular with a radius of R mm. A maximum distance between the projection of the guide cotton 3 on the atomizing plane 41 and the center of the atomizing plane 41 is L mm, and a minimum distance between the projection of the guide cotton on the atomizing plane and the center of the atomizing plane 41 is G mm, 0.3≤G/R≤L/R≤0.9. Through the pure essential oil passage, a projection area of the guide cotton 3 on the atomizing plane 41, that is, a contact area between the guide cotton 3 and the atomizing plane 41, is limited. In this way, the pure essential oil is more evenly spread on the atomizing plane 41, thereby enhancing the atomization effect and improving the atomization efficiency.

In some improvement solutions of the present disclosure, the one end of the guide cotton 3 can also be provided at a point of wave nodes of the ultrasonic atomization sheet 4. Since the guide cotton 3 is provided at the point of wave nodes of the ultrasonic atomization sheet 4, the vibration interference of the guide cotton 3 on the ultrasonic atomization sheet is reduced, such that the ultrasonic atomization sheet 4 can achieve optimal amplitude. In addition, since the guide cotton 3 is provided at the point of wave nodes of the ultrasonic atomization sheet 4, it can be prevented from burning.

In some improvement solutions of the present disclosure, an atomization temperature of the ultrasonic atomization sheet 4 is T° C., 80≤T≤160. An experiment shows that the viscosity of the pure essential oil decreases with the increase of temperature, and the ultrasonic atomization sheet 4 is easier to atomize a low-viscosity pure essential oil. Table 1 shows viscosity changes of a water-based oil at different temperatures.

TABLE 1
Viscosities of water-based pure essential
oil at different temperatures

| Temperature (° C.) | Viscosity (mPa · s) |
|---|---|
| 10 | 1.308 |
| 20 | 1.002 |
| 30 | 0.7978 |
| 40 | 0.6531 |
| 50 | 0.5471 |
| 60 | 0.4658 |
| 70 | 0.4044 |
| 80 | 0.3550 |
| 90 | 0.3150 |
| 100 | 0.2822 |

It can be seen from the above table that a higher temperature leads to a lower viscosity of the pure essential oil. In an embodiment of the present disclosure, the temperature of the ultrasonic atomization sheet 4 is controlled to quickly raise the temperature of the pure essential oil, thereby reducing the viscosity of the pure essential oil. Specifically, in the present disclosure, a heating rate of the ultrasonic atomization sheet 4 is controlled to increase the temperature of the ultrasonic atomization sheet 4 in a short period of time. The high temperature of the ultrasonic atomization sheet 4 can quickly heat the pure essential oil transported through the pure essential oil passage. By increasing the temperature of the pure essential oil, the viscosity of the pure essential oil is reduced, thereby reducing the difficulty of atomizing the pure essential oil with the ultrasonic atomization sheet 4. In addition, when the temperature of the pure essential oil increases, the molecular activity of the pure essential oil is enhanced, and the molecular force and surface tension of the pure essential oil are reduced, thereby accelerating the atomization of the pure essential oil, reducing the delay time of atomization start, and improving the user experience.

In some improvement solutions of the present disclosure, the guide cotton 3 includes a first guide cotton 31 and a second guide cotton 32 connected to each other. The first guide cotton 31 is attached to the atomizing plane 41, and the second guide cotton 32 extends through the housing 2 to the pure essential oil chamber 11. The first guide cotton 31 is made of a high temperature resistance material. Due to the atomization of the pure essential oil through a high temperature, as the use and atomization time increase, the first guide cotton 31 tightly attached to one end of the ultrasonic atomization sheet 4 will be burnt by the high temperature generated during the operation of the ultrasonic atomization sheet 4, resulting in poor atomization efficiency and poor atomization effect. Therefore, the present disclosure uses the first guide cotton made of a high temperature resistance material to prevent damage to the pure essential oil passage caused by the high temperature generated by the ultrasonic atomization sheet 4, thereby stabilizing the long-term pure essential oil supply to the surface of the ultrasonic atomization sheet 4. Specifically, the first guide cotton 31 can be made of a glass fiber, a porous ceramic, or other high temperature resistance guide material.

The second guide cotton 32 is made of a quick guide material that can quickly transfer the pure essential oil to the first guide cotton 31, ensuring stable pure essential oil supply. Specifically, the second guide cotton 32 can be made of a pure cotton material.

In some improvement solutions of the present disclosure, the guide cotton 3 can also contact the ultrasonic atomization sheet through a porous ceramic or other material as an intermediate medium to achieve pure essential oil transfer. Specifically, in the present disclosure, when the porous ceramic is used as the intermediate medium, due to its high temperature resistance, it can prevent the high temperature of the ultrasonic atomization sheet 4 from burning the guide cotton 3, thereby reducing the difficulty of selecting the guide cotton 3.

In some improvement solutions of the present disclosure, the opening 12 of the bottle body 1 is oriented upwards, and the housing 2 covers the opening 12 from top to bottom and is screwed to the bottle body 1. A bottom part of the housing 2 is provided with an air hole 23 that communicates the atomizing chamber 21 with the pure essential oil chamber 11. When the pure essential oil is transported up to the housing 2 through the second guide cotton 32 in the bottle body 1, a negative pressure is generated in the pure essential oil chamber 11 due to the reduction of the pure essential oil. The negative pressure will cause an internal pressure of the pure essential oil chamber 11 to be lower than an external air pressure, making it impossible to continuously guide the pure essential oil. Therefore, the air hole 23 communicated with an internal space of the pure essential oil chamber 11 is provided at the bottom part of the housing 2, such that the air pressure inside the pure essential oil chamber 11 is balanced with the air pressure outside the bottle body 1, achieving a continuous pure essential oil guiding effect. In addition, pure essential oil accumulation may occur at the bottom part of the atomizing chamber 21 after long-term atomization. The air hole 23 allows accumulated pure essential oil to flow back into the pure essential oil chamber 11, achieving the reuse of the pure essential oil.

In other improvement solutions of the present disclosure, the second guide cotton 32 is hollow. Since the second guide cotton 32 is hollow, air exchange can be carried out inside the second guide cotton 32 to balance the air pressure inside the pure essential oil chamber 11 with the air pressure outside the bottle body 1, achieving a continuous pure essential oil transfer effect.

In some improvement solutions of the present disclosure, the housing 2 is further provided therein with an air chamber 24. The housing 2 is provided with an air inlet 25. The air inlet 25 is communicated with the air chamber 24. The atomizing chamber 21 is communicated with the atomization outlet 22 through the air chamber 24. The atomizing plane 41 is oriented towards the air chamber 24. The air enters from the air inlet 25, mixes with a pure essential oil mist in the air chamber 24, and then blows out from the atomization outlet 22, alleviating the problem of the atomizer, that is, poor atomization effect caused by the fluctuation of the pure essential oil mist.

In some improvement solutions of the present disclosure, a cross-section of a wall of the air chamber 24 is circular to avoid airflow obstruction, rebound, and other impacts inside the air chamber 24. Specifically, the air chamber 24 can be a circular chamber.

In some improvement solutions of the present disclosure, the atomization outlet 22 is provided at a top part of the housing 2. The air inlet 25 is provided at a side wall of the housing 2, and the air inlet 25 and the atomization outlet 22 are respectively arranged on left and right sides of the housing 2. The air inlet and the atomization outlet 22 form convection, facilitating the air to enter from the air inlet 25 and then circulate in the air chamber 24 to fully mix with the pure essential oil mist before blowing out.

Overall, the embodiment of the present disclosure provides a pure essential oil atomization device. The ultrasonic atomization sheet 4 directly atomizes the pure essential oil. The pure essential oil atomization device needs to avoid direct contact between the pure essential oil and the ultrasonic atomization sheet 4, so as to avoid the problem of poor atomization efficiency and poor atomization effect caused by uneven pure essential oil supply. For this purpose, the guide cotton 3 serves as a pure essential oil passage to guide the pure essential oil in the pure essential oil chamber 11 to come into contact with the atomizing plane 41. The design enhances the atomization effect, improves atomization efficiency, effectively solves problems such as leakage and noise during use, and prevents the supply of the pure essential oil passage from being affected by a negative pressure.

The above are only preferred implementations of the present disclosure. It should be noted that several improvements and replacements may further be made by a person of ordinary skill in the art without departing from the technical principle of the present disclosure, and such improvements and replacements should also be deemed as falling within the protection scope of the present disclosure.

1. A pure essential oil atomization device, comprising a bottle body, a housing, a guide cotton, and an ultrasonic atomization sheet, wherein
the bottle body is provided therein with a pure essential oil chamber, and is provided with an opening communicated with the pure essential oil chamber; the housing is detachably connected to the bottle body, and covers the opening; the housing is provided therein with an atomizing chamber, and is provided with an atomization outlet communicated with the atomizing chamber; the ultrasonic atomization sheet is provided in the atomizing chamber, and comprises an atomizing plane; and the guide cotton comprises one end attached to the atomizing plane and surrounding a center of the atomizing plane, and the other end extending through the housing to the pure essential oil chamber.

2. The pure essential oil atomization device according to claim 1, wherein the atomizing plane is circular with a radius of R mm; and a maximum distance of L mm and a minimum distance of G mm between projection of the guide cotton on the atomizing plane and the center of the atomizing plane, 0.3<G/R≤L/R≤0.9.

3. The pure essential oil atomization device according to claim 1, wherein projection of the guide cotton on the atomizing plane surrounds the center of the atomizing plane, and a central angle of an arc of the projection is N°, 180≤N≤360.

4. The pure essential oil atomization device according to claim 1, wherein an atomization temperature of the ultrasonic atomization sheet is T° C., 80≤T≤160.

5. The pure essential oil atomization device according to claim 1, wherein the guide cotton comprises a first guide cotton and a second guide cotton connected to each other; the first guide cotton is attached to the atomizing plane, and the second guide cotton extends through the housing to the pure essential oil chamber; the first guide cotton is made of a high temperature resistance material; and the second guide cotton is hollow.

6. The pure essential oil atomization device according to claim 1, wherein the guide cotton is provided at a point of wave nodes of the atomizing plane.

7. The pure essential oil atomization device according to claim 1, wherein the opening of the bottle body is oriented upwards; the housing covers the opening from top to bottom and is screwed to the bottle body; and a bottom part of the housing is provided with an air hole that communicates the atomizing chamber with the pure essential oil chamber.

8. The pure essential oil atomization device according to claim 1, wherein the housing is further provided therein with an air chamber, and is further provided with an air inlet communicated with the air chamber; the atomizing chamber is communicated with the atomization outlet through the air chamber; and the atomizing plane is oriented towards the air chamber.

9. The pure essential oil atomization device according to claim 8, wherein a cross-section of a wall of the air chamber is circular.

10. The pure essential oil atomization device according to claim 9, wherein the atomization outlet is provided at a top part of the housing; the air inlet is provided at a side wall of the housing; and the air inlet and the atomization outlet are respectively arranged on left and right sides of the housing.
